(19) United States
(12) Patent Application Publication
(10) Pub. No.: US 2007/0190541 A1
(43) Pub. Date: Aug. 16, 2007

(54) Microarray substrate, methods of manufacturing and use

(76) Inventors: Jong-myeon Park, Seoul (KR); Sang-hyun Peak, Seoul (KR)

Correspondence Address: CANTOR COLBURN, LLP, 55 GRIFFIN ROAD SOUTH, BLOOMFIELD, CT, 06002, US

(21) Appl. No.: 11/523,459

(22) Filed: Sep. 19, 2006

(30) Foreign Application Priority Data
Dec. 20, 2005 (KR) 10-2005-0126263

(51) Int. Cl. C12Q 1/68; C12M 1/34

(52) U.S. Cl. 435/6; 435/287.2

(57) ABSTRACT

A microarray substrate comprising a functionalized (poly)ethyleneglycol compound coated on a solid support having a polyanhydride surface is disclosed. Methods of manufacturing and using the substrate are also disclosed.

### CROSS-REFERENCE TO RELATED PATENT APPLICATION

This application claims priority from Korean Patent Application No. 10-2005-0126263, filed on Dec. 20, 2005, in the Korean Intellectual Property Office, the disclosure of which is incorporated herein in its entirety by reference.

### BACKGROUND

1. Field of the Invention

The present invention relates to a microarray substrate and a method of manufacturing the substrate. More particularly, the present invention relates to a microarray substrate in which a functionalized (poly)ethyleneglycol compound is coated on a solid support having a polyanhydride surface, and a method of manufacturing the substrate.

2. Description of the Related Art

A microarray refers to a high-density array of predetermined molecules immobilized in predetermined regions of a substrate. The microarray can be classified as a polynucleotide microarray, a protein microarray, etc. The term “polynucleotide microarray” refers to a high-density array of two or more polynucleotides immobilized on a substrate in which each of the two or more polynucleotides is immobilized on different predetermined regions of the substrate. Microarrays are well known in the art. Examples of microarrays are disclosed in U.S. Pat. No. 5,445,934 and U.S. Pat. No. 5,744,305, the disclosures of which are incorporated herein in their entireties by reference.

The immobilization of polynucleotides on a solid substrate can be achieved by direct synthesis of polynucleotides on a solid substrate or by immobilization of previously synthesized polynucleotides on predetermined regions of a solid substrate. Illustrative methods for manufacturing such polynucleotide microarrays are disclosed in U.S. Pat. No. 5,744,305, U.S. Pat. No. 5,143,854, and U.S. Pat. No. 5,424,186, the disclosures of which are incorporated herein in their entireties by reference. A spotting method has been widely used for the immobilization of biomolecules on a solid substrate by covalent attachment of biomolecules on the solid substrate. For example, a method of immobilizing biomolecules on a solid substrate which has been widely used includes: activating a surface of the solid substrate with a nucleophilic functional group (e.g., an amino group), coupling biomolecules (e.g., polynucleotides) activated with a good leaving group to the surface-activated solid substrate, and removing unreacted reactants.

In addition, a microarray using hydrophilic polyethyleneglycol has been reported. For example, U.S. Patent Publication No. 2003-0108917A1 discloses a method of manufacturing a microarray wherein probe polynucleotides are immobilized on a hydrogel composed of a star-like polyethyleneglycol derivative having an epoxy end-functional group.

### SUMMARY OF THE INVENTION

The present invention provides a microarray substrate that enhances the immobilization efficiency of a probe biomolecule and reduces non-specific binding of a target biomolecule, e.g. a protein, on the substrate.

Disclosed herein is a microarray substrate comprising a functionalized (poly)ethyleneglycol compound coated on a solid support having a surface modified with a polyanhydride.

Also disclosed herein is a method of manufacturing a microarray substrate, the method including: (a) obtaining a solid support comprising a surface comprising an amino group-containing compound; (b) immobilizing a polyanhydride on a surface by reacting the polyanhydride with the amino group-containing compound; and (c) coating the polyanhydride-immobilized surface with a functionalized (poly)ethyleneglycol such that the functionalized (poly)ethyleneglycol reacts with the immobilized polyanhydride.

Also disclosed herein are a microarray comprising a probe biomolecule immobilized on the above-described microarray substrate and a method of analyzing a target biomolecule using the microarray.

The microarray substrate of the present invention enhances the immobilization efficiency of a probe biomolecule and blocks non-specific binding of a target biomolecule, or other components of a sample, to the microarray substrate. Therefore, after a probe biomolecule is immobilized on the microarray substrate, no further treatment of the microarray substrate is required, thereby ensuring process simplicity and cost-effectiveness in chip production and biomolecule assay. For example, a crude PCR sample can be directly applied to a microarray comprising the microarray substrate.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates immobilization of poly(ethylene-alt-maleic anhydride) on a solid support coated with gamma-aminopropyltriethoxysilane (GAPS);

FIG. 2 illustrates functionalizing a star-like polyethyleneglycol (PEG) with an amino group;

FIGS. 3A through 3D are ¹H NMR spectra of a compound of Formula 1 which is a starting compound for functionalizing PEG with an amino group (FIG. 3A), compounds of Formulae 2 and 3 which are intermediates in the functionalization of the PEG (FIGS. 3B and 3C), and a compound of Formula 4 which is the product amino group functionalized PEG (FIG. 3D), respectively;

FIG. 4 illustrates coating an amino-functionalized PEG on a solid support having a poly(ethylene-alt-maleic anhydride) surface;

FIG. 5A shows fluorescence image data obtained after a control microarray 1 and a test microarray 1 according to the present invention are hybridized with target DNAs and FIG. 5B illustrates fluorescence intensity data obtained after the hybridization of FIG. 5A (fluorescence intensity is expressed as relative fluorescence units (RFU));

FIG. 6 illustrates fluorescence intensity data obtained after FITC-labeled BSA is applied to a test substrate according to the present invention and a control substrate (fluorescence intensity is expressed as RFU); and

FIG. 7 shows fluorescence image data obtained after a control microarray 2 and a test microarray 2 according to the present invention, which are manufactured without performing a process for protecting an unreacted amino group on a substrate after probe immobilization, are hybridized with a target DNA sample which is not purified after amplification.

### DETAILED DESCRIPTION OF THE INVENTION

The present invention will now be described more fully with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

The present invention provides a microarray substrate comprising a functionalized (poly)ethyleneglycol compound coated on a solid support surface modified with a polyanhydride.

In the present invention, the material of the solid support is not particularly limited provided that it is a solid phase material capable of providing a surface. For example, the solid support may be a plastic substrate made of polyethylene, polypropylene, polystyrene, polyurethane, or polyolefin; a glass substrate; a silicone wafer; or a modified substrate thereof. In the present invention, the surface of the solid support may have a functional group capable of acting as a hydrogen bond donor, such as an amino group, a thiol group, or a hydroxy group. Such a functional group may already be present on the surface of the solid support itself due to the intrinsic characteristics of the material of the solid support or the functional group may be incorporated onto the surface of the solid support using a chemical or physical treatment (e.g., coating).

In an embodiment, the surface of the solid support is coated with an amino group-containing compound. The amino group-containing compound may be, for example, gamma-aminopropyltriethoxysilane (GAPS), gamma-aminopropyldiethoxysilane (GAPDES), or an aminohexyl group, but the present invention is not limited to the illustrated examples. Preferably, the surface of the solid support may be coated with GAPS.

The polyanhydride used herein may be poly(ethylene-graft-maleic anhydride), poly(isobutylene-alt-maleic anhydride), poly[(isobutylene-alt-malemide)co-isobutylene-alt-maleic anhydride], etc. Poly(ethylene-alt-maleic anhydride) is preferred. The poly(ethylene-alt-maleic anhydride) may have a molecular weight from 128 to 10,000,000, and most preferably from 100,000 to 500,000.

In the present invention, the surface of the solid support modified with the polyanhydride is coated with a functionalized (poly)ethyleneglycol compound. Here, the (poly)ethyleneglycol compound may be functionalized with an amino group, a thiol group, a hydroxy group, an epoxy group, or an ester group. The amino group is preferred. The (poly)ethyleneglycol compound may have a molecular weight from 60 to 10,000,000, preferably from 200 to 1,000,000, and more preferably from 200 to 10,000.

The (poly)ethyleneglycol compound may be a star-like or linear molecule.

The present invention also provides a method of manufacturing the microarray substrate. The method comprises: (a) obtaining a solid support comprising a surface comprising an amino group-containing compound; (b) immobilizing a polyanhydride on the surface of the solid support by reacting the polyanhydride with the amino group-containing compound; and (c) coating the polyanhydride-immobilized surface with a functionalized (poly)ethyleneglycol.

In the method of manufacturing the microarray substrate, the solid support may be obtained by purchasing a commercially available solid support comprising a surface comprising an amino group-containing compound or by preparing the solid support by incorporating an amino group-containing compound onto an unmodified solid support using a chemical or physical treatment (e.g., coating).

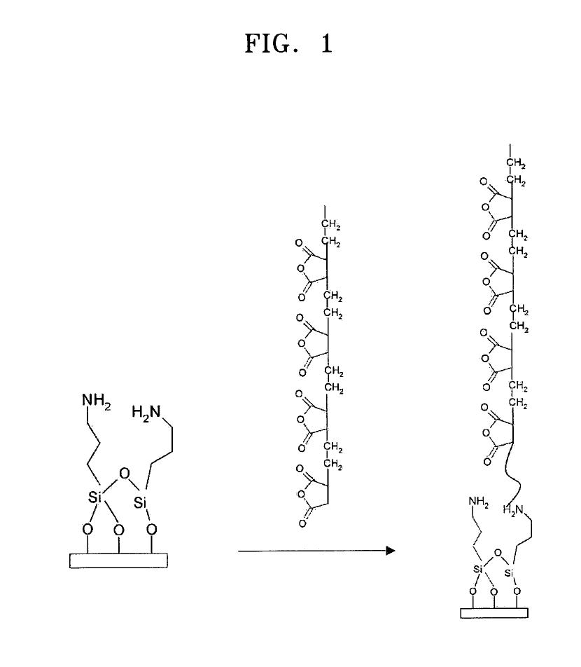

After obtaining the solid support comprising a surface comprising the amino group-containing compound, the polyanhydride is immobilized on the surface of the solid support (see FIG. 1). Generally, the covalent immobilization of the polyanhydride on the surface of the solid support is achieved by reacting the polyanhydride with the amino group present on the surface of the solid support.

After immobilizing the polyanhydride on the surface of the solid support, the solid support is surface-coated with the functionalized (poly)ethyleneglycol. The functionalized (poly)ethyleneglycol may be amino-functionalized (poly)ethyleneglycol. The amino-functionalized (poly)ethyleneglycol can be synthesized using any method commonly known in the art. (Poly)ethyleneglycol having as many amino groups as possible is preferred. Coating is performed such that the functionalized (poly)ethyleneglycol reacts with the immobilized polyanhydride. For example, an amino-functionalized (poly)ethyleneglycol reacts with the polyanhydride surface of the polyanhydride-immobilized solid support. In detail, an anhydride group of the polyanhydride reacts with the amino group of the amino-functionalized (poly)ethyleneglycol. As a result, many (poly)ethyleneglycols, amino groups, and carboxyl groups are exposed on the surface of the microarray substrate of the present invention.

The microarray substrate manufactured by the above-illustrated method has many amino-groups on its surface, and thus, enhances the immobilization efficiency of probe biomolecules. Furthermore, under the pH condition for probe-target hybridization, the (poly)ethyleneglycols and carboxyl groups on the surface of the microarray substrate prevent non-specific adsorption of target biomolecules to substrate regions other than the substrate regions having immobilized probe biomolecules. Additionally, the (poly)ethyleneglycols and carboxyl groups on the surface of the microarray substrate prevent non-specific adsorption of proteins, or other components of a sample, to the microarray substrate. The minimal non-specific adsorption characteristics of the microarray substrate of the present is invention ensure process simplicity and cost-effectiveness in chip production and biomolecule assay. Thus, for example, for a polynucleotide microarray comprising the microarray substrate disclosed herein, a hybridization process and a hybridization assay can be directly performed on a sample with the microarray without performing any post-treatment (e.g., background capping) of the substrate after probe immobilization on the substrate. Similarly, no further purification of a sample after cell lysis or PCR is required prior to performing a hybridization process and assay with a polynucleotide microarray comprising the microarray substrate disclosed herein.

The present invention also provides a microarray comprising a probe biomolecule immobilized on the microarray substrate and a method of analyzing a target biomolecule using the microarray.

In the present invention, a biomolecule may be selected from the group consisting of a nucleic acid, a protein, a polysaccharide, and a combination thereof. A nucleic acid is preferred. The nucleic acid may be DNA or RNA. In the present invention, the probe biomolecule immobilized on the microarray substrate is generally a biomolecule capable of specifically reacting with a target biomolecule. For example, when a nucleic acid is used as the probe biomolecule, the probe biomolecule can hybridize with a target nucleic acid having a nucleotide sequence complementary to the nucleotide sequence of the probe biomolecule or it can bind to a protein that binds specifically to a recognition sequence present in the probe biomolecule. When a protein is used as the probe biomolecule, the probe biomolecule can specifically interact with a target protein or a target nucleic acid through an antigen-antibody interaction, a ligand-receptor interaction, or an enzyme-substrate interaction. When a polysaccharide is used as the probe biomolecule, the probe biomolecule can specifically interact with a protein (e.g., lectin) or antibody recognizing the polysaccharide. The microarray according to the present invention can be used for various assays using a detection system capable of detecting the specific interaction between the probe biomolecule and the target molecule and an assay system capable of analyzing the detection result.

In the present invention, the concentration of the probe biomolecule to be immobilized to the microarray substrate may vary according to reaction conditions or the type of desired data. Thus, the concentration of the probe biomolecule is not particularly limited. In an embodiment of the present invention, when DNA is used as the probe biomolecule, the probe biomolecule may be used in the concentration of 20 to 100 μM, but the present invention is not limited thereto.

In the present invention, the immobilization of the probe biomolecule on the microarray substrate can be performed by any conventional method used for manufacturing a DNA or protein microarray. For example, a microarray can be manufactured using a photolithographic method. According to a photolithographic method, a polynucleotide microarray can be manufactured by repeatedly exposing a predetermined surface region of a substrate coated with a monomer protected by a removable protecting group to an energy source to remove the protecting group and coupling the deprotected monomer with another monomer protected by the removable protecting group. In this case, immobilization of a polynucleotide on the polynucleotide microarray can be achieved by synthesizing a polynucleotide by extending monomers of the polynucleotide one by one. Alternatively, a previously synthesized polynucleotide can be immobilized in a predetermined region (which is also called “spotting”).

Hereinafter, the present invention will be described more specifically with reference to the following working examples.

### EXAMPLE 1

### Preparation of Microarray Substrate According to the Present Invention and Control Substrate

(1) Manufacturing of Microarray Substrate According to the Present Invention

(a) Immobilization of Polyanhydride on Surface of Silicone Wafer Coated with GAPS

In order to manufacture a microarray substrate according to the present invention, first, a GAPS-coated silicone wafer (LG Siltron, Korea) was immersed in a solution of 200 mM (on a repeat unit basis) of poly(ethylene-alt-maleic anhydride) (molecular weight: 100,000 to 500,000) in N-methyl-2-pyrrolidone (NMP) at room temperature for one hour, washed with acetone and ethanol, and dried under vacuum (see FIG. 1).

(b) Preparation of Amino-Functionalized Polyethyleneglycol (PEG)

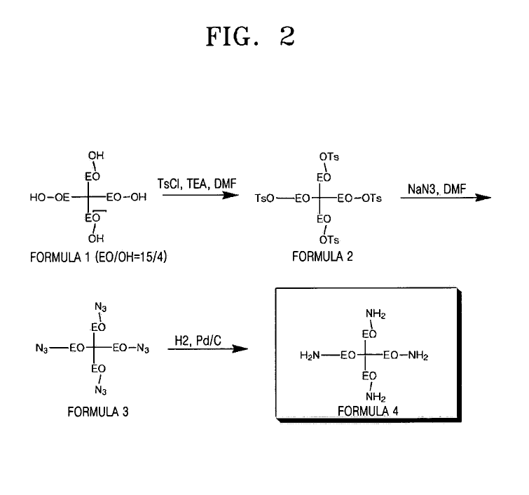

An amino-functionalized PEG compound wherein an amino group was incorporated into a PEG backbone was prepared as follows. The preparation method is illustrated in FIG. 2.

(i) Preparation of Compound of Formula 2

A star-like PEG (with an ethylene oxide (EO)/hydroxyl (OH) ratio=15/4) (1 eq.), a compound of Formula 1 used as a starting material (see FIG. 2), was incubated in the presence of triethylamine (TEA) (5 g, 6 eq.), dimethylformamide (DMF) (20 ml), and tosyl chloride (TsCl) (7.1 g, 6 eq.) at 120° C. for 3 hours. Reaction and disappearance of the starting material, PEG, was identified by thin layer chromatography (TLC) (elution solvent: 10% methanol in CHCl₃).

The reaction solution was diluted with methylene chloride (10 ml), transferred to a separatory funnel, and extracted with water to remove the DMF and methylene chloride. Then, solvent was removed using a rotary evaporator and the residue was purified by flash column chromatography. The flash column chromatography was performed by pressurizing a column packed with silica gel, loading the solvent-free residue on the silica gel, and pushing the elution solvent (10% methanol/90% chloroform) with compressed air. As a result, a compound of Formula 2 (FIG. 2) was obtained.

(ii) Preparation of Compound of Formula 3

The compound of Formula 2 (1 eq.) and sodium azide (NaN₃) (10 eq.) were incubated in DMF in the presence of TEA (10 eq.) at 100° C. overnight. Reaction and disappearance of the compound of Formula 2 was identified by TLC (elution solvent: 8% ethanol in CHCl₃).

The reaction solution was diluted with methylene chloride (10 ml), transferred to a separatory funnel, and extracted with water to remove DMF and methylene chloride. Then, solvent was removed using a rotary evaporator and the residue was purified by flash column chromatography as described above to give a compound of Formula 3.

(iii) Preparation of Compound of Formula 4

The compound of Formula 3 (5 g) was incubated in methanol (20 ml) in the presence of H₂ and a Pd/C catalyst (10 eq.). Reaction and disappearance of the compound of Formula 3 was identified by TLC (elution solvent: 8% methanol in CHCl₃) (Rf=0).

The reaction solution was filtered through a Celite pad and solvent was removed using a rotary evaporator to give a compound of Formula 4.

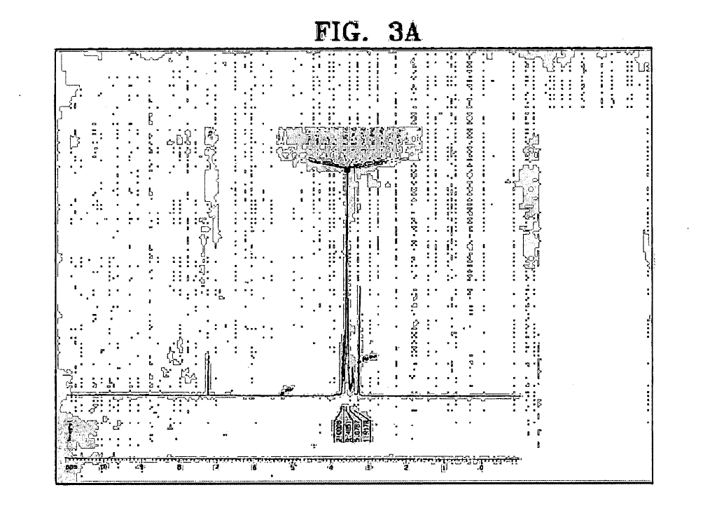

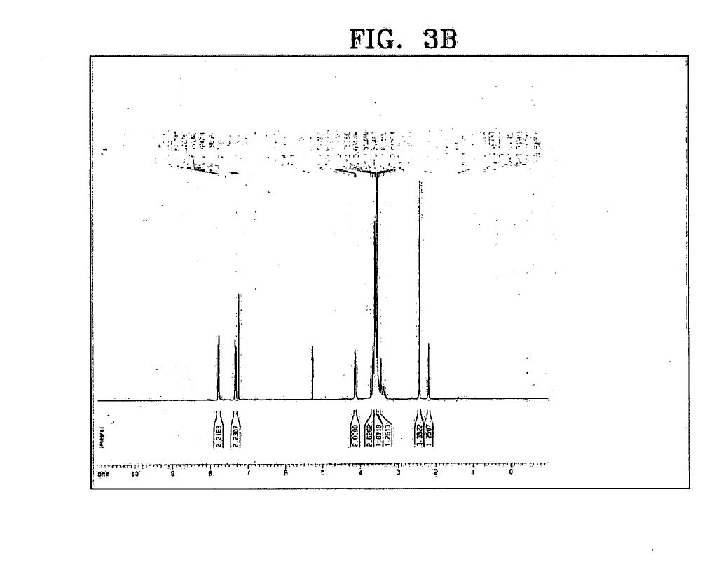

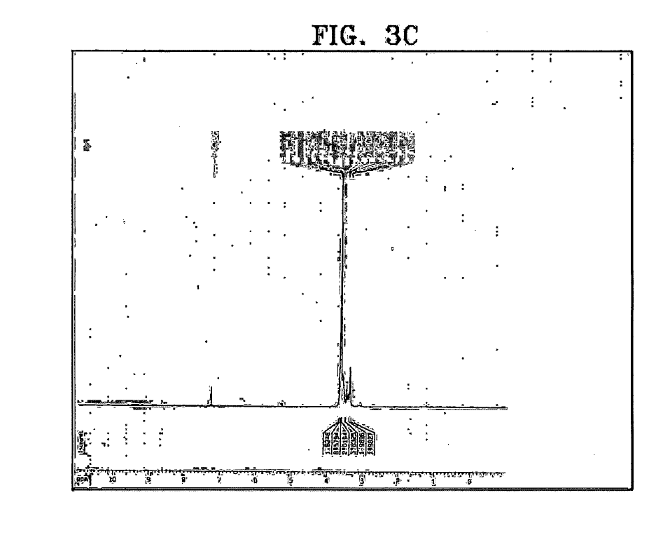

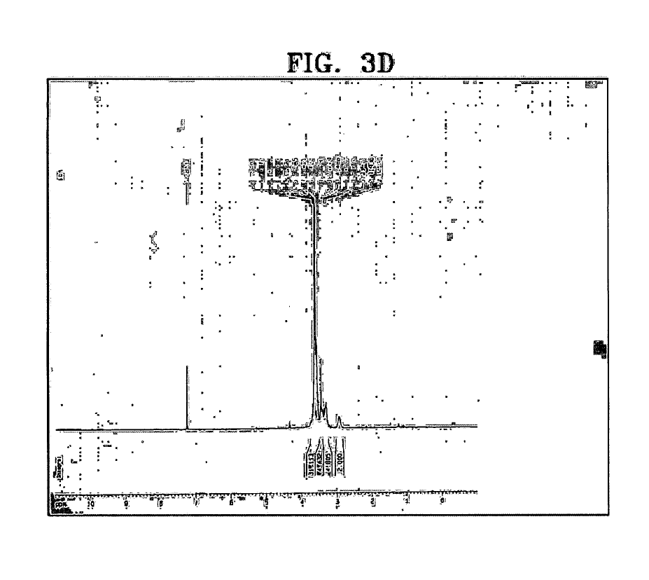

The compounds of Formulae 1 through 4 were characterized by NMR in a chloroform solvent using a Bruker 500 MHz ¹H NMR. The spectra of the four compounds are shown in FIGS. 3A through 3D, respectively.

(c) Coating of Polyanhydride-Immobilized Silicone Wafer with Amino-Functionalized PEG

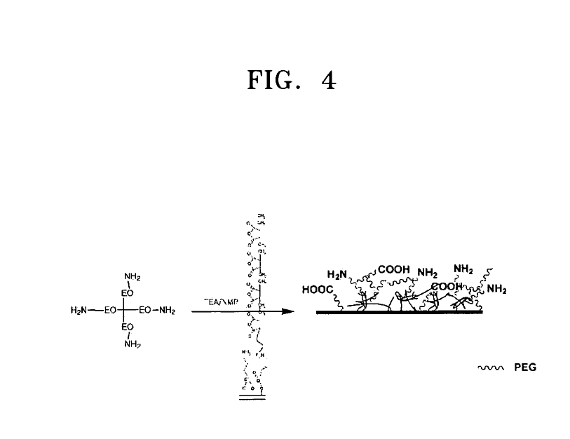

The polyanhydride-immobilized silicone substrate of (a) was immersed in a solution of the amino-functionalized PEG of (b) (200 mM) and water (300 mM) in NMP to induce hydrolysis, washed with ethanol and acetone, and dried, to give a microarray substrate according to the present invention (hereinafter, referred to as “test substrate”) (see FIG. 4).

(2) Control Substrate

In the following working examples, a GAPS-coated silicone wafer was used as a control substrate.

### EXAMPLE 2

### Manufacturing of Polynucleotide Microarray Wherein Probe DNA is Immobilized on Substrate

A polynucleotide microarray wherein 5′-end functionalized DNA was arranged in two or more groups of spots of the test substrate prepared in (1) of Example 1 was manufactured as follows.

First, a spotting solution was prepared. The composition of the spotting solution was as follows: 50% formamide, 25% a solution of 9 mM PEG (MW: 10,000) in NaHCO₃ (0.1M, pH 10), and 25% a solution containing probe DNA (SEQ ID NO: 1) having an amino (NH₂) group at the 5′-end. The final concentration of DNA molecules was 20 μM in distilled water.

The spotting solution thus prepared was spotted on the test substrate using a Pix5500 spotter (Cartesian), and incubated at 70° C., 40% humidity, for one hour, in a constant temperature and humidity chamber so that the probe DNA was immobilized on the test substrate. After termination of the reaction, the test substrate was washed with distilled water, incubated with anhydrous succinic acid (blocking agent) to protect an unreacted amino group, washed with ethanol, and spin-dried to obtain a polynucleotide microarray immobilized with the probe DNA, which was designated as “test microarray 1”.

Additionally, a “control microarray 1” was manufactured in the same manner as above using a GAPS-coated silicone wafer as the control substrate.

### EXAMPLE 3

### Evaluation of Immobilization Efficiency of Probe DNA

In Example 3, hybridization of target DNA to the probe DNA immobilized on the test microarray 1 manufactured in Example 2 was performed. The hybridization results were detected to thereby determine the immobilization efficiency of the probe DNA on the test microarray 1.

An oligonucleotide (SEQ ID NO: 2) having —NH₂-Cy3 at the 5′-end was used as a target DNA.

16 μl of the target DNA (100 pM molecule) was placed in a 1.5 ml tube, vortexed for 10 seconds, and centrifuged.

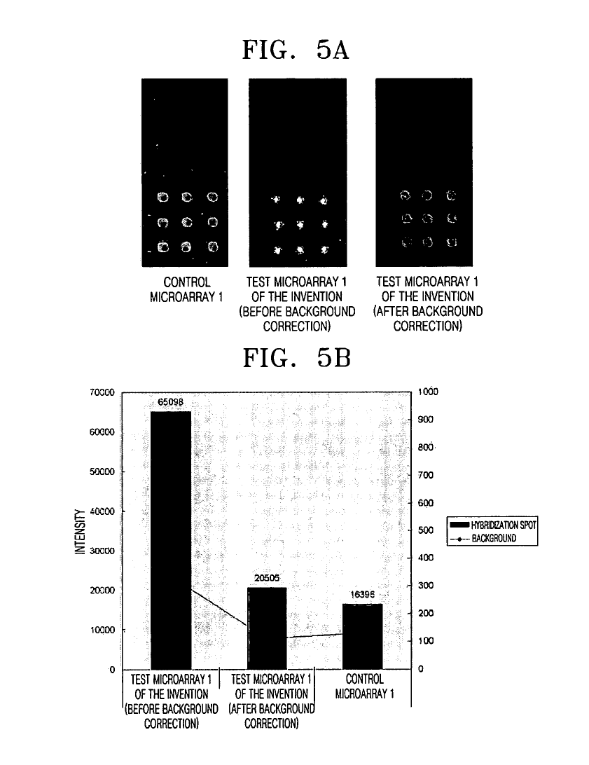

The target DNA was denatured in a 94° C. heating block for 5 minutes and placed on ice. In this state, 16 μl of a hybridization buffer (a solution of NaH₂PO₄H₂O 138 g, NaCl 876 g, 0.5M EDTA 200 ml, and 10N NaOH 100 ml) was added to the target DNA to reach a total volume of 32 μl. The reaction solution was vortexed for 10 seconds, centrifuged for 10 seconds, and added to the test microarray 1 or to the control microarray 1. The test microarray 1 and the control microarray 1 were incubated at 42° C. for one hour, washed with first a washing solution I (1×SSPET) and then a washing solution II (3×SSPET) for 5 minutes each, and dried. Fluorescence image data were acquired using a GenePix 4000B scanner (Axon Instruments) and analyzed using GenePix Pro software (Axon Instruments, Union City, Calif.). The fluorescence image data and the fluorescence intensity data are shown in FIGS. 5A and 5B, respectively. The fluorescence intensity was observed at 532 nm (PMT560).

Referring to FIGS. 5A and 5B, the immobilization efficiency of the probe DNA of the test microarray 1 before background correction was more than 300% higher than that of the control microarray 1. The immobilization efficiency of the probe DNA of the test microarray 1 after background correction was more than 30% higher than that of the control microarray 1.

### EXAMPLE 4

### Non-Specific Binding Test of Protein on Substrate According to the Present Invention

500 μl of FITC-labeled bovine serum albumin (BSA) (standard protein) in aqueous solution (1 mg/ml) was added to the test substrate and the control substrate prepared in Example 1. The test substrate and the control substrate were incubated in a covered chamber at room temperature for 2 minutes and washed with distilled water. Fluorescence intensity from the test substrate and the control substrate was measured.

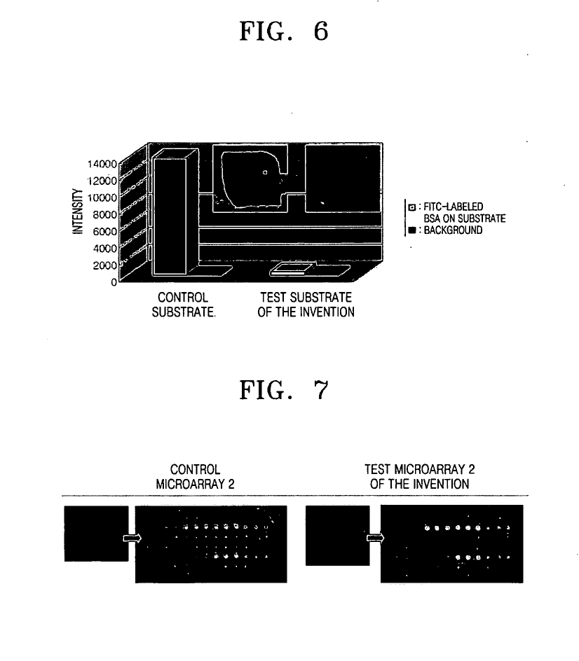

The fluorescence intensity was measured at 532 nm(PMT560) using a GenePix 4000B scanner (Axon Instruments) to acquire fluorescence images. The fluorescence images were analyzed using GenePix Pro software (Axon Instruments, Union City, Calif.). The results are presented in Table 1 below and FIG. 6.

TABLE 1

| | Control substrate | Test substrate |
|---|---|---|
| FITC-labeled BSA | 13924.93 | 465.37 |
| Background | 110 | 213 |

As shown in Table 1 and FIG. 6, non-specific binding of the protein on the test substrate of the invention was significantly reduced (at least 96% reduction) as compared to the control substrate.

### EXAMPLE 5

### Non-Specific Binding Test of Target DNA Under the Conditions of No Purification After PCR and No Post-Treatment After Probe Immobilization

A test microarray 2 of the invention and a control microarray 2 were manufactured in the same manner as in Example 2 except that three types of oligonucleotides (SEQ ID NOS: 3 through 5) were used as probe DNAs and, unlike in Example 2, no process for protecting unreacted amino groups with a blocking agent was performed after probe DNA immobilization.

Target DNAs were designed to have DNA sequences corresponding to 16S rRNA and 23S rRNA of Staphylococcus aureus which was a type of respiratory bacteria.

The target DNA corresponding to 16S rRNA was amplified by PCR using oligonucleotides having SEQ ID NOS: 6 and 7 as forward and reverse primers. The target DNA corresponding to 23S rRNA was amplified by PCR using oligonucleotides having SEQ ID NOS: 8 and 9 as forward and reverse primers.

5′-ends of the amplified target DNAs were labeled with Cy5. Then, the reaction solutions containing the Cy5-labeled target DNAs were hybridized to test microarray 2 and control microarray 2 without further purification. The hybridization was performed in the same manner as in Example 3.

Fluorescence images from test microarray 2 and control microarray 2 before and after the hybridization were acquired at 532 nm using a GenePix 4000B scanner (Axon Instruments). The fluorescence images were analyzed using GenePix Pro software (Axon Instruments, Union City, Calif.). The results are presented in Table 2 below and FIG. 7. Background emission was determined for the regions of the substrate of test microarray 2 or control microarray 2 that did not have immobilized probe DNA to hybridize to the Cy5-labeled target DNA.

TABLE 2

| | Control microarray 2 | | Test microarray 2 | |
|---|---|---|---|---|
| | Before | After | Before | After |
| | hybridization | hybridization | hybridization | hybridization |
| Background | 61.11 | 154.85 | 63.31 | 82.56 |
| Increase (%) | | 154 | | 30 |

As shown in Table 2 and FIG. 7, when performing the hybridization with no protection of the unreacted amino groups after probe immobilization, background emission from the control microarray 2 was increased by more than 150% as compared to that before the hybridization, whereas background emission from the test microarray 2 was increased by only 30% as compared to that before the hybridization.

These results show that with respect to a microarray of the present invention, even when no process is performed to block unreacted amino groups exposed on the surface of the microarray substrate after probe immobilization, non-specific binding of target DNA to the microarray substrate is significantly reduced compared to the control microarray.

As described above, for a microarray substrate of the present invention, the immobilization efficiency of a probe biomolecule is enhanced and non-specific binding of a target biomolecule or a protein or other contaminant on the microarray substrate is blocked. Thus, after a probe biomolecule is immobilized on the microarray substrate, no further post-treatment of the microarray substrate is required, and a crude PCR sample can be directly applied to the microarray substrate, thereby ensuring process simplicity and cost-effectiveness in chip production and biomolecule assay.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. While the present invention has been particularly shown and described with reference to exemplary embodiments thereof, it will be understood by those of ordinary skill in the art that various changes in form and details may be made therein without departing from the spirit and scope of the present invention as defined by the following claims. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A microarray substrate comprising a functionalized (poly)ethyleneglycol compound coated on a solid support having a surface modified with a polyanhydride.

2. The microarray substrate of claim 1, wherein the (poly)ethyleneglycol compound is functionalized with an amino group, a thiol group, a hydroxy group, an epoxy group, or an ester group.

3. The microarray substrate of claim 2, wherein the (poly)ethyleneglycol compound is functionalized with the amino group.

4. The microarray substrate of claim 1, wherein the (poly)ethyleneglycol compound is a star-like or linear molecule.

5. The microarray substrate of claim 4, wherein the (poly)ethyleneglycol compound has a molecular weight of 60 to 10,000,000.

6. The microarray substrate of claim 1, wherein the polyanhydride is poly(ethylene-alt-maleic anhydride).

7. The microarray substrate of claim 6, wherein the poly(ethylene-alt-maleic anhydride) has a molecular weight of 128 to 10,000,000.

8. The microarray substrate of claim 1, wherein the solid support is made of a material selected from the group consisting of silicone, glass, and a plastic material.

9. The microarray substrate of claim 8, wherein the surface of the solid support is coated with an amino group-containing compound, a thiol group, or a hydroxyl group-containing compound.

10. The microarray substrate of claim 9, wherein the surface is coated with the amino group-containing compound; and the amino group-containing compound is gamma-aminopropyltriethoxysilane, gamma-aminopropyldiethoxysilane, or aminohexyl.

11. The microarray substrate of claim 10, wherein the amino group-containing compound is gamma-aminopropyltriethoxysilane.

12. A method of manufacturing a microarray substrate, the method comprising:
(a) obtaining a solid support comprising a surface comprising an amino group-containing compound;
(b) immobilizing a polyanhydride on the surface by reacting the polyanhydride with the amino group-containing compound; and
(c) coating the polyanhydride-immobilized surface with a functionalized (poly)ethyleneglycol such that the functionalized (poly)ethyleneglycol reacts with the immobilized polyanhydride.

13. The method of claim 12, further comprising functionalizing an end of a (poly)ethyleneglycol with an amino group.

14. The method of claim 12, wherein the functionalized (poly)ethyleneglycol is a star-like or linear molecule.

15. The method of claim 12, wherein the functionalized (poly)ethyleneglycol has a molecular weight of 60 to 10,000,000.

16. The method of claim 12, wherein the polyanhydride is poly(ethylene-alt-maleic anhydride).

17. The method of claim 16, wherein the poly(ethylene-alt-maleic anhydride) has a molecular weight of 128 to 10,000,000.

18. The method of claim 12, wherein the solid support is made of a material selected from the group consisting of silicone, glass, and a plastic material.

19. The method of claim 12, wherein the amino group-containing compound is gamma-aminopropyltriethoxysilane, gamma-aminopropyldiethoxysilane, or aminohexyl.

20. A microarray comprising a probe biomolecule immobilized on the microarray substrate of claim 1.

21. The microarray of claim 20, wherein the probe biomolecule is a nucleic acid.

22. A method of analyzing a target biomolecule, comprising
binding a target biomolecule to the probe molecule of the microarray of claim 20.


SEQUENCE LISTING (9 sequences)

SEQ ID NO: 1 - DNA, 15 bp, Artificial Sequence
  probe
tgttctcttgtcttg

SEQ ID NO: 2 - DNA, 15 bp, Artificial Sequence
  target DNA
caagacaagagaaca

SEQ ID NO: 3 - DNA, 22 bp, Artificial Sequence
  probe
acggagttacaaaggacgacat

SEQ ID NO: 4 - DNA, 22 bp, Artificial Sequence
  probe
aaaggacgacattagacgaatc

SEQ ID NO: 5 - DNA, 25 bp, Artificial Sequence
  probe
aacatatgtgtaagtaactgtgcac

SEQ ID NO: 6 - DNA, 20 bp, Artificial Sequence
  primer
yccakactcctacgggaggc

SEQ ID NO: 7 - DNA, 22 bp, Artificial Sequence
  primer
gtgccagcagyygcggtaatac

SEQ ID NO: 8 - DNA, 22 bp, Artificial Sequence
  primer
tagcatatcagaaggcacaccc

SEQ ID NO: 9 - DNA, 23 bp, Artificial Sequence
  primer
aatgttgtctctcttgagtggat